(19) United States
(12) Patent Application Publication
(10) Pub. No.: US 2021/0007898 A1
(43) Pub. Date: Jan. 14, 2021

(54) Methods, Materials, Devices and Systems for Treating Injuries to Central Nervous System

(71) Applicants: William Loudon, Scottsdale, AZ (US); Spyro Mousses, Scottsdale, AZ (US); Sharief Taraman, Scottsdale, AZ (US)

(72) Inventors: William Loudon, Scottsdale, AZ (US); Spyro Mousses, Scottsdale, AZ (US); Sharief Taraman, Scottsdale, AZ (US)

(21) Appl. No.: 16/978,971

(22) PCT Filed: Mar. 4, 2019
(86) PCT No.: PCT/US2019/020626

§ 371 Date: Sep. 8, 2020

Related U.S. Application Data
(60) Provisional application No. 62/638,948, filed on Mar. 5, 2018.

(51) Int. Cl. A61F 13/00; A61F 13/12; A61N 5/06; A61L 15/44

(52) U.S. Cl. CPC A61F 13/00068; A61L 2300/256; A61N 5/0613; A61F 13/00063; A61L 15/44; A61N 2005/0647; A61N 2005/0659; A61N 2005/0626; A61N 2005/0632; A61N 2005/0664; A61F 2013/0017; A61F 2013/00182; A61F 2013/00285; A61L 2300/414; A61L 2300/406; A61L 2400/12; A61F 13/12

(57) ABSTRACT

There is provided improved therapy methods and apparatus for treating a patient's brain and spinal cord. The apparatus further relates to a flexible surgical balloon or cap-like device comprising one or more material layers that may have an antimicrobial element. The therapy apparatus may include portals for introducing diagnostic monitoring and/or therapeutic devices, and delivery of therapeutic drug delivery systems. Certain embodiments of the invention have an output emission area positioned to irradiate a portion of the brain with an efficacious power density and wavelength of light for diagnostic and/or therapeutic purposes.

### FIELD OF THE INVENTION

The present invention relates in general to methods and apparatuses for brain and spinal surgery and phototherapy, and more particularly, to novel apparatuses and methods for brain and spinal surgery and to phototherapy of central nervous system tissue.

### BACKGROUND

Neurological disorders ranked as the leading cause of disability-adjusted life-years in 2015 and the second-leading cause of deaths comprising 16.8% of global deaths (Global, regional, and national burden of neurological disorders during 1990-2015: a systematic analysis for the Global Burden of Disease Study 2015).

Traumatic brain injury, or TBI, is a significant medical problem worldwide. (Naeser MA, Hamblin MR. (2015). Within the United States, three traumatic brain injuries occur every minute, and more than 5,000,000 Americans live with TBI-related disabilities with an annual cost of $60 to $76.5 billion. (Maas AI, Menon DK. (2012) Traumatic brain injury: rethinking ideas and approaches. Lancet; 11:12-13.)

The most prevalent manifestation of TBI comprising 75 percent of cases are classified as mild TBI (mTBI), defined by loss of consciousness (LOC) lasting <30 minutes (or no LOC), and characterized by a period of altered mental status, such as amnesia or confusion, lasting no more than 24 hours. In the remaining 5 to 22 percent of patients, representing moderate or severe brain injury, persistent cognitive deficits profoundly impair their quality of life and their ability to contribute to society . (Naeser MA, Hamblin MR. (2015). Traumatic brain injury: A major medical problem that could be treated using transcranial, red/near-infrared LED photobiomodulation. Photomed Laser Surg; 33(9): 443-446). The most common causes of TBI arise from incidents related to falls, motor vehicle accidents, and assaults, but also extend to athletics and military combat, including blunt and/or penetrating injuries inflicted on rescue workers and victims of terrorist-related attacks. (Langlois JA, Rutland-Brown W, Wald MM. (2006). The epidemiology and impact of traumatic brain injury: a brief overview. J Head Trauma Rehabil. 21(5):375-378).

Another clinically significant form of brian injury is stroke. Stroke represents the second leading cause of death worldwide with the absolute number of people who have a stroke every year, stroke survivors, related deaths, and the overall global burden of stroke continuing to increase (Valery L Feigin, Lancet. 2014 Jan. 18; 383(9913): 245-254. Global and regional burden of stroke during 1990-2010: findings from the Global Burden of Disease Study 2010). In 2013 approximately 10.3 million people suffered a stroke. In 2015, it is estimated that 42.4 million people had survived a stroke. About half of people who have had a stroke live less than one year. https://en.wikipedia.org/wiki/Stroke accessed Dec. 6, 2017.

Research into brain injury has made great progress in clarifying the pathophysiological mechanisms. TBI consist of a primary injury resulting from direct biomechanical forces and subsequent secondary insults that play an important role in potentially compounding the degree of brain damage with resultant increase risk of death following TBI. (Cheng G, et al. (2012). Regardless of the mechanism of the inciting insult, after sustaining a clinically significant brain injury, the resultant injured brain can be subdivided into three populations. The first subpopulation is represented by non-viable tissue. This subpopulation is not amenable to revitalization. If sufficient brain tissue is killed by the primary insult, representing a critical percentage of the total brain, all hope of functional recovery for the patient is lost and the diagnosis of Brain Death is determined. The second subpopulation is represented by injured “at risk” tissues. This population is perched between functional recovery in one extreme and cell death in the other. This “at risk” population is uniquely sensitive to secondary injury resulting from secondary insults. The therapeutic goal and the aim of all research for treating brain injury focuses on maiximizing the repair and subsequent functional recovery of injured brain. Finally, the third group is composed of healthy tissues spared from the inciting primary insult. However, this third population remains susceptible to subsequent injury and conversion into tissues of the second or even the first subpopulations mentioned above as a result of the downstream effects of secondary insults. The overall clinical outcome of a brain injured patient is determined by the success of salvaging the injured “at risk” injured population while protecting the third subpopulation from subsequent injury. The mechanism of secondary cell death, designated apoptosis, is mediated by highly complex, genetically orchestrated processes resulting in “programmed cell death”. Optimal salvage of injured tissues must optimize cellular repair mechanisms while simultaneously blocking the triggers which invoke the irreversible initiation of cellular apoptosis. Mitochondria in traumatic brain injury and mitochondrial-targeted multipotential therapeutic strategies. Br J Pharmacol, 167: 699-717.) The imbalance between higher energy demands (in the form of ATP) required for repair of cell damage, opposed by decreased ATP production (led by mitochondrial dysfunction) further aggravates potential for survival and meaningful recovery from brain injury. At the cellular level, the main cause of secondary injury cascades (following the primary insult of acute brain injury cascades) is cell damage that is centered in the mitochondria. Excitotoxicins, Ca2+ overload, reactive oxygen species (ROS), Bcl-2 family, caspases and apoptosis-inducing factor (AIF) are the main participants in mitochondria-centered cell damage following TBI. (Cheng G, et al. (2012). Mitochondria in traumatic brain injury and mitochondrial-targeted multipotential therapeutic strategies. Br J Pharmacol, 167: 699-717.)

Mitochondria may represent the evolutionary remains of aerobic bacteria, that infected and internalized themselves within anaerobic protoeukaryotic cell about a billion years ago, possessing a separate genome and providing a symbiotic relationships offering a broad range of metabolic functions essential for cell homoeostasis. (Hagberg H, et al. (2014). Mitochondria: hub of injury responses in the developing brain. Lancet Neurol; 13: 217-32.) Mitochondria are dynamic double-membrane bound organelles that are responsible for ATP generation, calcium regulation, and the biosynthesis of aminoacids, lipids, and nucleotides. (Green DR, Galluzzi L, Kroemer G. (2011). Mitochondria and the autophagy-inflammation-cell death axis in organismal aging. Science; 333: 1109-12.) Electron flow through the electron transport chain generates a proton gradient across the inner mitochondrial membrane, which drives the production of ATP by ATP synthase (Hagberg H, et al. (2014). Mitochondria: hub of injury responses in the developing brain. Lancet Neurol; 13: 217-32.)

Mitochondrial fusion and fission, biogenesis, and mitophagy are processes that are crucial for functional recovery of neurons after injury; impairment of fusion and fission is implicated in progression of Alzheimer's disease and Huntington's disease (Song W, et al. (2011). Mutant Huntingtin binds the mitochondrial fission GTPase dynamin-related protein-1 and increases its enzymatic activity. Nat Med; 17: 377-82; Seo AY, et al. (2010). New insights into the role of mitochondria in aging: mitochondrial dynamics and more. J Cell Sci; 123: 2533-42.) Mitochondria also play a critical role in the regulation of innate immune responses, and are commanders of inflammation, synaptic development and connectivity, and repair, with far-reaching implications for the brain's susceptibility to damage and central nervous system (CNS) capacity to repair injury. (Hagberg H, et al. (2014). Mitochondria: hub of injury responses in the developing brain. Lancet Neurol; 13: 217-32.)

The major purpose of oxidative phosphorylation in mitochondria is to produce ATP. ATP is continuously produced almost exclusively by the oxidation of glucose, and is the energy carrier fueling most cellular homeostatic mechanisms. When ATP supply is insufficient, homeostatic mechanisms deteriorate, intracellular concentration of calcium increases, and neural apoptosis (programmed cell death) is inevitable. (Verweij BH, et al. (2000). Improvement in mitochondrial dysfunction as a new surrogate efficiency measure for preclinical trials: dose-response and time-window profiles for administration of the calcium channel blocker Ziconotide in experimental brain injury. J Neurosurg 93: 829-834.) Prior research efforts in patients with TBI have been deficient, insofar as they only focused on optimizing the delivery of oxygen and glucose to the injured brain environment in an attempt to maintain the ATP supply, while failing to address any underlying impairment of mitochondrial function.

At the cellular level, two initiating events related to energy depletion and Ca2+ homeostasis are of particular importance in the response to primary injury. The first is an ‘ischemia-like’ pattern that is characterized by direct tissue damage and impaired regulation of cerebral blood flow (CBF) and metabolism, within which ATP stores are depleted, and failure of energy-dependent membrane ion pumps occurs. (Cheng G, et al. (2012). Mitochondria in traumatic brain injury and mitochondrial-targeted multipotential therapeutic strategies. Br J Pharmacol, 167: 699-717.)

The second event is characterized by nerve terminal membrane depolarization along with excessive release of excitatory neurotransmitters (i.e. glutamate, aspartate), activation of NMDA, AMPA and voltage-dependent Ca2+ and Na+ channels, in turn releasing additional Ca2+ from intracellular stores and producing abnormally high levels of free intracellular and mitochondrial Ca2+. (Cheng G, et al. (2012). Mitochondria in traumatic brain injury and mitochondrial-targeted multipotential therapeutic strategies. Br J Pharmacol, 167: 699-717.) Traumatic disruption of the blood-brain barrier may further flood the interstitial space with supraphysiologic concentrations of excitoxins such as glutamate. It has been demonstrated in both animal models and human subjects that TBI results in mitochondrial dysfunction, which is characterized by impaired ATP production and calcium ion regulation. (Xiong Y, et al. (1997). Mitochondrial dysfunction and calcium perturbation induced by traumatic brain injury. J Neurotrauma; 14: 23-34; Verweij, BH, et al. (2000). Impaired cerebral mitochondrial function after traumatic brain injury in humans. J Neurosurg; 93: 815-820.) The consecutive Ca2+ overload leads to self-digesting (catabolic) intracellular processes that involve overproduction of free radicals, activation of apoptotic cell death signaling pathways and up-regulation of inflammatory mediators. (Cheng G, et al. (2012). Mitochondria in traumatic brain injury and mitochondrial-targeted multipotential therapeutic strategies. Br J Pharmacol, 167: 699-717.)

Together, these events lead to membrane degradation of vascular and cellular structures, and ultimately trigger neural cell death. As the ‘power plant of the cell’, ATP production via oxidative phosphorylation is the primary function of mitochondria, and CA2+ is the characteristic stimulatory signal for activation of numerous mitochondrial enzymes (Graier et al., 2007). Several studies in recent years have indicated that mitochondria play a pivotal role in neuronal cell survival. Mitochondrial dysfunction is considered to be an early event in CNS injury that can cause apoptotic neuronal cell death.

Past attempts at using red or near-infrared irradiation therapy via non-invasive methods have proven to be unfruitful, with the most pertinent example being Photothera's NEST-III clinical trial. This attempt at non-invasive near-infrared (NIR) therapy for traumatic brain injury, despite its success in irradiating the entire brain in the small-animal models used in the preclinical studies, could not sufficiently penetrate the thicker humans skulls, and therefore could not penetrate deeply enough to beneficially affect motor function. (PhotoThera Inc. (2013). Efficacy and safety trial of transcranial laser therapy within 24 hours from stroke onset (NEST-3). https://clinicaltrials.gov/show/NCT01120301.

See also U.S. Pat. Nos. 6,312,451, 6,537,304, 6,918,922, 7,288,108, 7,303,578, 7,309,348, 7,316,922, 7,344,555, 7,534,255, 7,575,589, 7,695,504, 7,848,035, and 8,025,687, all incorporated by reference herein in their entireties.

Some preclinical and clinical results of mitochondria-targeted therapy show promise. Mitochondria-targeted multipotential therapeutic strategies offer new hope for the successful treatment of TBI and other acute brain injuries. (Cheng G, et al. (2012). Mitochondria in traumatic brain injury and mitochondrial-targeted multipotential therapeutic strategies. Br J Pharmacol, 167: 699-717.)

Brain injury commonly results in the acute onset of cerebral edema (brain swelling), the accumulation of fluid in, and resultant swelling in, the brain. Current standard of care for neurosurgery in the treatment of severe brain swelling resulting from brain injury includes the clinical option of a surgical procedure called a decompressive craniectomy to accommodate said cerebral edema.

Decompressive craniectomy can be defined as the removal of a large area of the skull to increase the potential volume of the cranial cavity. (Hutchinson P, Timofeev I, Kirkpatrick P. (2007).

Surgery for Brain Edema. Neurosurg Focus; 22(5): E14.) Brain swelling following TBI and other varieties of cerebral insults contributes to the above-mentioned secondary cascades of brain injury, and detrimentally affects long-term functional outcome. Decompressive craniectomy can serve to alleviate some of the pressure on the brain and reduces these secondary injury cascades. (Timofeev, I., et al. (2012). Decompressive craniectomy-operative technique and perioperative care. Advances and technical standard in neurosurgery. Volume 28. New York: Spring-erWienNewYork, pp. 121). Primary (prophylactic or early) Decompressive craniectomy is ideally performed soon after the pathological insult, and is aimed at preventing or mitigating the adverse effects related to the developing brain edema, whereas secondary decompression is defined as a postponed removal of the bone flap, initially left in situ and/or enlargement of an initial craniectomy, motivated by subsequent medically refractory brain swelling. (Bell RS, et al. (2010). Early decompressive craniectomy for severe penetrating and closed head injury during wartime. Neurosurg Focus; 28: E1.) Even though decompression has the potential advantage of limiting the damage associated with escalation of brain swelling and, therefore, secondary damage at an early stage, protocol-driven decompressive craniectomy could minimize the number of operations and associated risks while still providing physiological protection against secondary insults, guided by neuromonitoring. (Timofeev, I., et al. (2012). Decompressive craniectomy-operative technique and perioperative care. Advances and technical standard in neurosurgery. Volume 28. New York: Springer Wien NewYork, pp. 121-136.)

The definitive relationship between pathologically elevated intracranial pressure and increased morbidity and mortality after BI has been conclusively validated in a number of cohort studies (Balestreri M, et al. (2006). Impact of intracranial pressure and cerebral perfusion pressure on severe disability and mortality after head injury. Neurocrit Care; 4: 8-13; for example). As a result, decompressive craniectomy represents one of the most effective surgical measures for controlling medically refractory ICP, and may be used as a preventive measure or as part of a therapeutic protocol.

The most commonly employed surgical operations include unilateral, bifrontal, and bilateral decompression; other approaches include bioccipital, circumferential, and “floating” or “hinge” craniotomy with in situ retention of a mobile bone flap (Stefini R, et al. (2007). Bi-occipital decompressive craniectomy in refractory post traumatic intracranial hypertension: first report of one case. Br J Neurosurg; 21: 527-31; Schmidt JH et al. (2007). Use of hinge craniotomy for cerebral decompression. Technical note. J Neurosurg 107: 678-82). In most cases, unilateral hemicraniectomy or wide bifrontal decompression are sufficient. (Timofeev, I., et al. (2012). Decompressive craniectomy-operative technique and perioperative care. Advances and technical standard in neurosurgery. Volume 28. New York: SpringerWienNewYork, pp. 121-136.)

A substantial body of evidence exists to suggest that too small a decompressive craniectomy leads to inadequate decompression, and therefore limits Intracranial pressure (ICP) control with potential development of an “external brain hernia”, or fungus celebri (brain herniation via the craniectomy window). (Honeybul S. (2010). Complications of decompressive craniectomy for head injury. J Clin Neurosci; 17: 430-35; Wagner S, et al. (2001). Suboptimum hemicraniectomy as a cause of additional cerebral lesions in patients with malignant infarction of the middle cerebral artery. J Neurosurg; 94: 693-96). Bearing in mind that inadequate craniectomy fails to serve its therapeutic purpose while creating additional complications, a large craniectomy with a minimum diameter of 12 cm has been recommended. (Timofeev, I., et al. (2012). Decompressive craniectomy-operative technique and perioperative care. Advances and technical standard in neurosurgery. Volume 28. New York: SpringerWienNewYork, pp. 121-136.)

The procedures for performing a decompressive craniectomy are as follows. The patient is placed under general anesthesia. Once the patient is positioned supine on the operating table, the head may be immobilized in a head clamp (i.e., a Mayfield Skull Clamp), and the hair is shaved from anterior hairline to 3-4 cm posterior to the coronal suture, extending to the level of zygomatic arch. (Timofeev, I., et al. (2012). Decompressive craniectomy-operative technique and perioperative care. Advances and technical standard in neurosurgery. Volume 28. New York: SpringerWien NewYork, pp. 121-136.) The caution area is marked on the surface anatomy, and the incision line marked, at which point local anesthetic containing a vasocontrictive agent (eg. Epinephrine) is infiltrated into the incision tract. (Quinn TM, et al. (2011). Decompressive craniectomy: technical note. Acta Neurol Scand; 123: 239-244.) It is at this point that the patient's head must be prepped and balloond in sterile fashion. (Quinn TM, et al. (2011). Decompressive craniectomy: technical note. Acta Neurol Scand; 123: 239-244.)

Following prepping and draping of the patient's head, the incision would be made as previously marked, and any major scalp bleeding stopped through the use of Rainey clips. (Quinn TM, et al. (2011). Decompressive craniectomy: technical note. Acta Neurol Scand; 123: 239-244.) The scalp would subsequently be easily detached from the periosteum by the loose areolar connective tissue, and the skin flap reflected and secured out of the way.

Burr holes would then be made in the skull to facilitate the use of a surgical drill (craniotome) which connects the burr holes and completes the bone cut. The dura mater, which encases the brain and spinal cord is detached from the skull epidurally. The bone flap is now removed from the skull. (Quinn TM, et al. (2011). Decompressive craniectomy: technical note. Acta Neurol Scand; 123: 239-244.) The dura mater is then opened, exposing the brain. This procedure is done with great trepidation, because if great brain swelling is encountered, once the dura is opened, brain tissue may herniate out through the dural opening, essentially choking the tissue and compressing the vasculature which has herniated through the opening. Upon completion of the surgery, the dural closure may be assisted by augmenting the closure, adding a piece of synthetic dura sewn into the opening to reduce restriction of volume from the dura.

It is at this point in time that it becomes important to ensure that the skin is closed in a watertight fashion in order to reduce the risk of infection. In the case of severe brain swelling, this presents a problematic limit, as it becomes necessary to forcefully pack the swollen brain back under the skin—a maneuver which usually portends death. In this respect, cerebrospinal fluid (CSF) drainage, hypertonic fluids, and pressors are all implemented in an attempt to reduce brain swelling, decrease intracranial pressure and optimize cerebral perfusion pressure (CPP). The instant invention solves these problems and removes the need to forcefully pack the swollen brain back under the skin.

The present invention provides a therapeutic alternative for the management of cerebral edema which involves severe morbidity rates at present.

Therapeutic hypothermia for acute brain injury is the intentional lowering of body temperature, with the objective of reducing tissue damage in the central nervous system. Techniques to induce and maintain hypothermia can be divided into two types: external and internal cooling methods. External measures include use of cooling blankets and local cooling using helmet devices. Despite their non-invasive nature, these methods have several disadvantages such as complex implementation, particularly in obese patients, high nursing requirements, intense skin vasoconstriction with resultant shivering, slow onset of the desired temperature and erratic temperature maintenance. Internal cooling methods that use central venous catheters to either infuse cool saline or directly to reduce the blood temperature by convection.

Cerebral therapeutic hypothermia has been developed as a strategy to mitigate secondary injury after a cerebral insult. Cerebral hypothermia is now considered a standard of care for an intervention of birthing complications resulting in hypoxic insults. Hypothermia has also been studied in mitigating brain injury during cardiac bypass. Finally, cerebral hypoperfusion has been studied in the setting of brain injury over the last several decades, with mixed results. It is not currently a recommended intervention in the current National AANS brain injury guidelines. Practical limitations to whole body cooling, or blood coolers, intended to drop cerebral temperatures to mild 35 or moderate 32 degrees F. hypothermia contribute to mixed outcome results.

The brain bubble disclosed herein offers clinically significant advantages over conventional whole-body and/or blood volume cooling strategies to achieve cerebral hypothermia. Brain Bubble irrigation solutions in all of their potential formulations, are amenable to cooling. Direct heat exchange on the surface of the brain without systemic cooling would greatly increase the efficacy and safety of cerebral hypothermia

Alzheimer's, Parkinson's and Huntington's diseases are all due to the abnormal accumulation of protein aggregates in the brain. Alzheimer' s disease and other dementias accounted for 40.2 to 52.7 million cases (Global, regional, and national burden of neurological disorders during 1990-2015: a systematic analysis for the Global Burden of Disease Study 2015).

Application of the above technique and invention in the case of severe neurological dysfunction in patients with abnormal accumulation of protein aggregates or toxins utilizing the portals available would allow for targeted removal of these aggregates.

The present invention provides a therapeutic alternative for the management of neurological disorders due to toxins and abnormal accumulation of protein aggregates which involves severe morbidity rates at present.

Spinal cord injury is an important cause of morbidity and mortality with an annual incidence of 10,000 to 12,000 cases in the United States (A global perspective on spinal cord injury epidemiology. Ackery A, Tator C, Krassioukov A, J Neurotrauma. 2004 October; 21(10): 1355-70). The United States National Institute of Neurological Disorders and Stroke (NINDS) now estimates that over $4 billion are spent annually on medical treatment alone for acute SCI and management of chronically debilitated patients (Kirshblum S, Campagnolo DI, DeLisa JA. Spinal cord medicine. Philadelphia: Lippincott Williams & Wilkins; 2002. p. 655 & A global perspective on spinal cord injury epidemiology. Ackery A, Tator C, Krassioukov A, J Neurotrauma. 2004 October; 21(10): 1355-70).

The current standard of care for acute spinal cord injury is medical therapy with steroids; however, there have been multiple studies investigating the role of surgical intervention compared to conservative and medical treatments, as well as concerning the optimal therapeutic window for surgical intervention. Surgery has the potential advantage of obtaining greater neurological recovery and facilitating earlier rehabilitation through decompression of the spinal cord and nerve roots, in addition to preventing further neurological deterioration and secondary damage following injury (Li Y, Walker CL, Zhang YP, Shields CB, Xu X-M. Surgical decompression in acute spinal cord injury: A review of clinical evidence, animal model studies, and potential future directions of investigation. Frontiers in biology. 2014; 9(2): 127-136).

The invention can be employed using a simple modification of classical spinal cord decompression technique. Depending on the anatomy at the location of injury, a skin incision is extended deep towards the bony architecture of the vertebral column encasing the spinal cord. Depending on the nature of the injury and location, adequate removal of bone to allow for decompression may destabilize the spine, requiring further surgery to recover necessary stability. After achieving bony decompression, the covering sheath (dura) surrounding the spinal cord is carefully opened being careful to not injure the spinal cord in the process. Further surgical intervention such as removal of blood clots, infection, foreign bodies (eg. bullets) may be performed at this time. With the goals of decompression and washout achieved, and a direct communication from the skin to the surface of the spinal cord achieved, the invention can be applied to the skin in the same fashion as the technique used for the head, and intervening volume filled with appropriate irrigation solution.

The present invention provides a therapeutic alternative for the management of spinal cord injury which involves severe morbidity rates at present.

### DETAILED DESCRIPTION OF THE INVENTION

According to certain embodiments of the instant invention, a surgical device comprising a flexble, sterile bag or covering may be applied to preoperatively prepared intact skin edges via combinations of adhesive such as colloidal tape and/or physical connectors (eg. staples) to achieve a water-tight seal. This seal can be reapplied as necessary during the useful timeframe of device placement.

The application procedure for the device would include a bicoronal (ear to ear) incision of skin and underlying soft tissues, with reflection of skin flaps both forward and backward to potentially expose frontal, temporal, parietal and occipital skull. Large portions of skull would be deliberatively removed according to surgical plan based upon patient's individual brain injury. Removed skull would be sterilely processed and stored for later surgical reimplantation. The sterile “bag”, “brain bubble” or “surgical neurological balloon” would then be attached.

The use of the flexible coverings according to the present invention allow for the nearly total exposure of the brain into a therapeutic, decompressive, monitored, sterile environment that is amenable to additional therapeutic interactions.

The surgical neurological balloons of the present invention provide a number of addition advantages as set forth below:

They have the ability to release an antimicrobial agent to a surgical wound over a period of time to reduce the risk of infection.

They provide additional protection to medical personnel working on a patient.

Embodiments of the present invention provide a surgical option for situations in which a physician encounters a medical condition in which pathological cerebral edema requires the brain to expand beyond the potential volume of the cranium or current surgical strategies for decompressive craniectomy for a sustainable period of time to allow the brain to heal inter-operatively of up to a year in duration.

They may be left in position on the body for extended periods of time which enables a long period of healing during which the brain can freely swell and subsequently contract without additional, secondary damage and enables the inclusion of portals for therapeutic access to the exposed brain tissue and thus may allow for continuous washout of the brain tissue.

In certain embodiments the surgical neurological balloons of the present invention resemble a swimming cap or an IV bag placed on the head.

In certain embodiments the devices according to the invention allow for continuous washout of the brain to remove accumulated proteins such as beta amyloids or prions, toxins, infectious agents such as viral, bacterial and fungal pathogens, as well as auto-antibodies.

Xie et al (Science. 2013 Oct. 18; 342(6156) incorporated herein in its entirety by reference) demonstrated the ability of increased interstitial fluid flows to increase the rate of beta-amyloid clearance from the brain. They postulate that the restorative role of sleep is the result of the clearance of accumulated neurotoxic waste products from the brain. Pathological substrates associated with neurodegenerative diseases, including β-amyloid (Aβ), α-synuclein, and tau, are present in the interstitial space surrounding cells of the brain but the brain lacks a conventional lymphatic waste removal system. Instead, cerebrospinal fluid (CSF) recirculates through the brain, interchanging with interstitial fluid (ISF) and removing interstitial proteins, including Aβ. These pathways were named the glymphatic system and is more effective during sleep when the interstitial space is increased by as much as 60%.

The glymphatic system is a recently discovered macroscopic waste clearance system that utilizes a unique system of perivascular channels, formed by astroglial cells, to promote efficient elimination of soluble proteins and metabolites from the central nervous system. (See Jessen et al. Neurochem Res. 2015 December; 40(12): 2583-2599-The Glymphatic System—A Beginner's Guide—incorporated herein by reference in its entirety)

In certain aspects of the present invention the portals are used to introduce equipment intended to “wash” the brain and remove accumulated neurotoxins. Washing fluids should generally not contain harsh detergents or unwanted proteases. In certain embodiments targeted antibodies may be used in the washing process. The antibodies may be attached to nanoparticles to facilitate the removal of unwanted, accumulated proteins. In certain embodiments coated magnetic beads may be also be used to support antibodies or other efficacious molecules.

In certain embodiments, cannabinoids may be provided to the brain through the ports in order to block the accumulation of intracellular AP by the activation of cannabinoid receptors. (Currais et al. npj Aging and Mechanisms of Disease (2016) 2,16012; doi:10.1038/npjamd. 2016.12; published online 23 Jun. 2016-incorporated in its entirety herein by reference).

In another embodiment, the portals allow for improved access to the exposed brain for improved diagnostics such as MRI, Doppler, blood flow, IR, perfusion, PET, CT, and spectroscopy methods.

The instant invention may be implemented at the time of decompressive craniectomy and opening of dura mater. After the surgical area has been isolated and treated with anti-microbial cleaning techniques, the sterile site is isloated with sterile towels and a surgical drapes, and an adhesive antimicrobial material may also be aplied to cover some or all of the patient's exposed skin. Said antimicrobial skin barriers, by way of their application to the surgical site, are capable of suppressing the spread of bacteria more effectively than through the use of topical antimicrobial agents alone. These agents typically are in the form of preoperative skin preps, surgical scrub tissues, washes, wound cleaners, lotions and ointments. Although such topical applications are effective for shorter periods of times, their efficacy is limited as a result of their limited delivery time.

In accordance with one embodiment of the present invention, a sheet material for use as a surgical neurological balloon is provided which contains an antimicrobial agent dispersed therein, capable of releasing the antimicrobial agent over a period of time. The incorporation of the antimicrobial material in the balloon offers a degree of protection to a patient against infections associated with surgery, as well as by the threat of contamination from the environment and/or medical personnel working on the patient.

In accordance with one embodiment of the present invention, the surgical balloon comprises a polyethylene terephthalate (PET) layer bonded to a polypropylene fiber layer, the surface of the PET layer wrapped with biological polypeptide antibacterial coatings by bonding.

In another embodiment, the surgical balloon comprises a synthetic flexible polymeric film having incorporated therein an antiseptically effective amount of broad-spectrum antimicrobial gent. In a particularly preferred embodiment, the polymeric material is polyethylene or polyurethane, and the antimicrobial agent is 5-chloro-2-(2,4-dichlorophenoxy)phenol.

In another embodiment, the present invention relates to a surgical balloon comprising a film of a synthetic polymeric material having incorporated therein an amount of a first antiseptically active broad-spectrum antimicrobial agent, and a pressure sensitive adhesive coated onto one surface of said film, said pressure sensitive adhesive optionally having incorporated there through a second antiseptically active broad-spectrum antimicrobial agent. The first and second antimicrobial agents may be different or may be the same. The latter inventive surgical balloon is able to provide timed release of the antimicrobial agents from both the balloon material itself and the adhesive, which is used to attach the balloon material to skin. The use of antimicrobial agents in both the balloon material and the adhesive further reduces the risk of infection to a patient and to medical personnel working on the patient.

In yet another embodiment, the present invention relates to a sheet material for use as a surgical balloon which releases an antiseptically active broad-spectrum antimicrobial agent when placed in contact with skin produced by a process comprising the steps of: providing a mixture of polymeric material and an antimicrobial agent in which said antimicrobial agent is uniformly dispersed in said polymeric material; and forming said mixture into thin film. The formation of the thin film is preferably accomplished by utilizing an extrusion technique.

Accordingly, it is an object of the present invention to provide a surgical balloon for the head capable of reducing the risk of infection to a patient.

It is a further object of the present invention to provide a surgical balloon capable of releasing an antiseptically active broad-spectrum antimicrobial agent to a patient when the balloon is placed in contact with skin.

It is yet a further object of the present invention to provide an adhesive associated with a surgical balloon wherein both the adhesive and the surgical balloon are able to release an antiseptically active broad-spectrum antimicrobial agent when placed in contact with skin. In certain embodiments the adhesive may be a colloidal tape.

It is an additional object of the present invention to produce a surgical balloon wherein the balloon is produced by forming a film of a polymeric material having dispersed therein an antimicrobial agent.

Neurosurgery standard of care for stroke, intracranial hemorrhage, and traumatic brain injury offers wide decompressive craniectomy to help control intracranial pressure and facilitate better cerebral perfusion pressure. Intracranial pressure monitor may be placed, ideally including a ventriculostomy, into the ventricle which can measure pressure and also drain CSF to relieve pressure. Additional diagnostic probes to directly measure interstitial paramaters such as the Licox pO2 oxygen sensor probe can be placed to monitor the penumbra (the fringe area of the brain injury—the tissue at risk).

At the time of surgical removal of the skull to increase intracranial volume, the dura mater which encases the brain and spinal cord is usually opened with a piece of synthetic dura sewn into the opening to reduce restriction of volume from the dura at the close of the procedure. This procedure is done with great trepidation because if great brain swelling is encountered, once the dura is opened, brain tissue may herniate out through the dural opening, essentially choking the tissue and compressing the vasculature which has herniated through the opening. Hypothetically, the larger the craniectomy, the larger the dural opening, which leads to more space being created with less risk to the brain.

Classically, the skin needs to be closed in a water tight fashion to reduce risk of infection. This represents a problematic limit with severe brain swelling, having to forcefully pack the swollen brain back under the skin, negating the potential benefits of the craniectomy—a maneuver which usually portends death. CSF drainage, hypertonic fluids, and pressors are all used to attempt to restrict brain edema, reduce pathologically elevated intracranial pressure, and maximize CPP.

Techniques to further increase the ability to expand the vault volume of the skull and to address malignant brain swelling as a result of brain injury has not been advanced in decades. Reversibly removing large sections of skull (decompressive craniectomy) and/or opening dura mater and augmenting its volume has been practiced by some, but these procedures do not fall under the standard of care. Clinical data suggests that sufficiently large decompressions performed within first 24 hours correlates with improved survival. Improved neurologic functional outcome is more difficult to statistically document.

If extreme brain swelling is encountered, craniectomy and dural augmentation fails to generate sufficiently increased vault volume forcing the brain to be compressed upon closing of the skin. In these extreme circumstances, the ability to leave the skin open and allow the brain to swell to its necessary extreme would be ideal if the exposed brain could be protected from desiccation and infection. Therefore, the application of a sterile, water-tight brain bag applied to the skull with multiple access ports to add and remove, perhaps circulate fluids such as synthetic CSF, antibiotics, hyperoxygenated fluids (oxygen carrying hydrocarbons are currently being clinically tested as synthetic blood replacements, under severe trauma conditions), and other therapeutic interventions presents a potential option in these situations.

In certain embodiments, the present invention is directed to surgical balloons generally and is particularly suited to balloons which employ polymeric substrates although numerous materials suitable herein are known to those skilled in the art. The inventive surgical balloon is applied to the patient at the site of the decompressive craniectomy, where a surgical incision is to be made. The surgical balloon is characterized by having incorporated in its substrate material an antiseptically effective amount of a broad-spectrum antimicrobial agent. When used, the antimicrobial agent migrates to the outer surface of the substrate where it is released to the patient. Once present upon the skin, the agent acts to inhibit bacterial growth and promote asepsis. As the agent is removed by the skin, it is replenished from the balloon.

The thin sheet substrate used as the surgical balloon may be selected from a number of materials. The substrate may be a woven or knitted fabric comprised of antimicrobial containing fibers or a nonwoven fabric, but a plastic or polymeric film, e.g., polyvinyl chloride, polypropylene polyethylene or polyurethane, is particularly preferred. The polymeric films may be continuous in that they have no openings, but the moisture vapor transmissive character of some suitable films are based on the permeability of the materials to moisture vapor. These films are generally impermeable to liquid water and to other liquids. Examples of polymeric substrates useful in the present invention are described in U.S. Pat. No. 3,645,835 to Hodgson and are commercially available.

The substrate, preferably a polymeric sheet, can be up to 75 microns in thickness. More preferably it is less than 40 microns and usually about 30 microns in thickness.

In certain preferred embodiments, the material should be sufficiently porous to allow oxygen to reach the wound, and sufficiently rigid to prevent wound overgrowth. Open cell polyester foams such as those employed in WoundVac systems are an example (see U.S. Pat. Nos. 7,198,046, and 5,645,081 which are incorporated herein by reference in their entirety).

In certain embodiments, the surgical neurological balloon may be a flexible plastic “container” similar to an IV bag fabricated from multilayer sheeting composed of combinations of polypropylene, polyamide and polyethylene. Administration connectors may be added. In certain embodiments, the surgical neurological balloons may be composed of polyolefin/polyamide co-extruded plastic wrapped with a protective plastic pouch composed of polyamide/polypropylene.

In certain embodiments, the materials used to make the surgical neurological balloons may be polyvinyl chloride (PVC). Ethylene vinyl acetate may be used in combination with antibiotics where there is no need to autoclave the balloons. Polypropylene-based neurological balloons, which are autoclavable, may be used in certain embodiments of the present invention.

Propyflex from Pactiv may be used in certain embodiments of the invention. Pactiv is a three-layer coextruded flat or tubular film consisting of polypropylene and styrene ethylene butylene styrene.

Copolyester ether may also be used. Polypropylene materials according to the invention will contain other materials added to make them more flexible and durable. Straight polypropylene is too brittle and stiff. Combination of polypropylene or other polyolefin resins to a copolyester ether, such as Ecdel elastomer, may be used in monolayer or multilayer films incorporating Ecdel elastomer. When used in multilayer films, Ecdel as the outer layer provides toughness, clarity, and flexibility.

Incorporated into and throughout the structure of the polymeric sheet may be an antiseptically active broad-spectrum antimicrobial agent which releases from the polymeric sheet upon contact with human skin. The antimicrobial agent functions to prevent bacterial growth at the site of incision and further functions to provide protection to medical personnel working on the patient.

A large number of antimicrobial agents are contemplated for use in the present invention. Non-limiting examples of such antimicrobial agents include:
a) metal salts, or like compounds with antibacterial metal ions, e.g., copper, mercury or silver, and optionally with additional nonmetallic ions of antibacterial properties;
b) typical antibiotics, e.g., neomycin, soframycin, bacitracin, polymyxin;
c) antibacterials such as chlorhexidine and its salts;
d) quaternary ammonium compounds, e.g., cetrimide, domiphen bromide, and polymeric quaternaries;
e) iodophors such as povidone iodine, and polyvinylpyrrolidone-iodine (PVP-I);
f) acridine compounds such as 9-aminoacridine; 3,6-diaminoacridine and 6,9-diamino-2-ethoxyacridine;
g) biguanidine compounds such as 1,6-di(4-chlorophenylbiguanido)hexane, dia-minohexylbiguanide, 1,6-di(aminohexylbiguanido)hexane; and polyhexamethylene-biguanide; and
h) 5-chloro-2-(2,4-dichlorophenoxy)phenol available under the name Microban® from Microban Products.

The antimicrobial agent is preferably present in the balloon in an amount of about 0.01% to about 25% by weight of substrate material, more preferably between about 1% and about 5% by weight.

For example, when producing a polyethylene surgical balloon having an antimicrobial dispersed therein by an extrusion technique, a blow film type extruder having a circular die is used to produce a balloon having a thickness of less than 75 microns. This technique involves extrusion of polyethylene feed through the circular die, followed by expansion, cooling, and collapsing of the blown bubble. In operation, the blown film is extruded through guiding devices into a set of pinch rolls which flatten it. An example of such a blown film extruder is disclosed in U.S. Pat. No. RE 28,600.

In an embodiment wherein a fabric balloon is produced, the antimicrobial agent is directly added to the fabric forming material such as the fiber from which the yearns and threads forming the fabric are formed. The resultant material is then formed into a balloon by knitting, in the case of a knitted balloon, or by utilizing process techniques known in the art to produce a nonwoven fabric.

It is additionally possible to incorporate an antimicrobial adhesive throughout the substrate. Antimicrobial agents which may be incorporated into the adhesive include those from groups (i)-(viii) as described above. One embodiment includes the antimicrobial agent 5-chloro-2-(2,4-dichlorophenoxy)phenol. In certain embodiments it is preferred to use a balloon having an antimicrobial agent dispersed therein to provide additional protection to the surgery patient.

To disperse the antimicrobial agent into the substrate, techniques such as mixing the antimicrobial agent directly into the substrate materials themselves or solvent evaporation techniques such as those disclosed in U.S. Pat. Nos. 4,310,509 and 4,643,181 are used. Solvent evaporated techniques typically involve forming an emulsion of the antimicrobial agent in a solvent, and mixing the emulsion into the substrate so that the antimicrobial agent is uniformly dispersed as a separate phase throughout the medium. The solvents used to form the emulsion may be a single type of solvent or a combination of solvents selected from water or water-soluble solvents such as methanol, ethanol, ethyl acetate, tetrahydrofuran and the like. Mixing of the emulsion typically occurs at low mixing rates, about 300 rpm, and at ambient temperatures.

In the embodiment when the antimicrobial agent is directly applied to the surgical balloon, the agent may be applied to the balloon in solution as an aqueous dispersion, as a hot melt, or by a transfer process using known techniques such as knife, roller-coating or curtain-coating methods. The transfer process is particularly preferred.

The inventive surgical balloon may also include additional materials to conform to the desired use, such as commercially available antistatic materials. An example of such an antistatic material is Electrosol S-1-X, manufactured by Alframine.

In certain embodiments, the surgical balloons of the present invention have ports, similar to an intravenous bag, through which not only antibiotics, saline, or therapeutic molecules but also NIR light emitting equipment can be introduced.

Novel therapies for management of TBI, emphasizing recovery of injured brain tissue, involve the use of red or near-infrared irradiation therapy (R/NIR, 630-1000 nm). (Fitzgerald M et al. (2013). Red/near-infrared irradiation therapy for treatment of central nervous system injuries and disorders. Rev. Neurosci.; 24(2): 205-226; Quirk BJ, et al. (2012). Near-infrared photobiomodulation in an animal model of traumatic brain injury: improvements at the behavioral and biochemical levels. Photomed. Laser Surg.; 30(9): 523-529.)

Photons in the R (red) and NIR wavelengths have the potential to improve subnormal, cellular activity of brain tissue that has been damaged by brain trauma. (Naeser, MA et al. (2014). Significant improvements in cognitive performance post-transcranial, red/near-infrared light-emitting diode treatments in chronic, mild traumatic brain injury: open-protocol study. J Neurotrauma; 31: 1008-1017.)

Two physiological changes associated with exposure of cells to red and NIR wavelengths of light are: (1) Increased production of adenosine triphosphate by the mitochondria; and (2) increased vasodilation/regional cerebral blood flow (rCBF). (Karu TI, Pyatibrat LV, Afanasyeva, NI. (2005). Cellular effects of low power laser therapy can be mediated by nitric oxide. Lasers Surg. Med. 36, 307-314.; Lane N. (2006). Cell biology: power games. Nature; 443: 901-903; Nawashiro H, et al. (2012). Focal increase in cerebral blood flow after treatment with near-infrared light to the forehead in a patient in a persistent vegetative state. Photomed. Laser. Surg.; 30: 231-233.)

The last enzyme complex of the electron transport chain within the mitochondrial membrane, cytochrome c oxidase, is a photo acceptor for red and NIR photons. (Tedford CE, et al. (2015). Quantitative analysis of transcranial and intraparenchymal light penetration in human cadaver brain tissue. Lasers Surg Med; 47: 312-322; Pitzschke A., et al. (2015). Red and NIR light dosimetry in the human deep brain. Phys Med Biol; 60: 2921-2937. When the mitochondria are exposed to red/NIR photons, nitric oxide is released and diffused outside the cell wall, promoting local vasodilation and increased blood flow. (Naeser, MA et al. (2014). Significant improvements in cognitive performance post-transcranial, red/near-infrared light-emitting diode treatments in chronic, mild traumatic brain injury: open-protocol study. J Neurotrauma; 31: 1008-1017.)

Mitochondrial ATP production is also increased, thereby eliciting improvements in cellular respiration, oxygenation, and function. (Naeser, MA et al. (2014). Significant improvements in cognitive performance post-transcranial, red/near-infrared light-emitting diode treatments in chronic, mild traumatic brain injury: open-protocol study. J Neurotrauma; 31: 1008-1017.)

Multiple studies performed on animal subjects have shown significantly improved recovery of motor and cognitive function following exposure to NIR low-level laser therapy when treated in the acute post-injury phase, with improved energy kinetics and decreased inflammation being suggested as possible mechanisms for acute neuroprotection. (Oron A., et al. (2007). Low-level laser therapy applied transcranially to mice following traumatic brain injury significantly reduces long-term neurological deficits. J. Neurotrauma; 24: 651-656; Khuman J., et al. (2012). Low-level laser light therapy improves cognitive deficits and inhibits microglial activation after controlled cortical impact in mice. J. Neurotrauma; 29: 408-417; Wu Q., et al. (2012). Low-level laser therapy for closed-head traumatic brain injury in mice: effect of different wavelengths. Lasers Surg. Med.; 44: 218-226; Xuan W., et al. (2013). Transcranial low-level laser therapy improves neurological performance in traumatic brain injury in mice: effect of treatment repetition regimen. PLoS One 8, e53454.) Application of R/NIR LED therapy has also improved executive function and verbal memory in two patients with chronic TBI, with one subject who had been on medical disability for five months prior to LED treatments returning to full-time employment after treatment. (Naeser, MA, et al. (2011). Improved cognitive function after transcranial, light-emitting diode treatments in chronic, traumatic brain injury: two case reports. Photomed. Laser Surg.; 29: 351-358.)

Conditions specific to the nervous system have shown improved recovery following R/NIR-IT, including retinal degeneration, CNS injury, and stroke. (Natoli R, et al. (2010) Gene and noncoding RNA regulation underlying photoreceptor protection: microarray study of dietary antioxidant saffron and photobiomodulation in rat retina. Mol Vis; 16: 1801-1822; Albarracin RS, Valter K. (2012). Treatment with 670-nm light protects the cone photoreceptors from white light-induced degeneration. Adv Exp Med Biol; 723: 121-128; Byrnes KR, et al. (2005). Light promotes regeneration and functional recovery and alters the immune response after spinal cord injury. Lasers Surg Med; 36: 171-185; Lapchak PA. (2012). Transcranial near-infrared laser therapy applied to promote clinical recovery in acute and chronic neurodegenerative diseases. Expert Rev Med Devices; 9: 71-83; Detaboada L, et al. (2006). Transcranial application of low-energy laser irradiation improves neurological deficits in rats following acute stroke. Lasers Surg Med; 38: 70-73; Oron A, et al. (2006). Low-level laser therapy applied transcranially to rats after induction of stroke significantly reduces long-term neurological deficits. Stroke; 37: 2620-2624; Moreira MS, et al. (2009). Effect of phototherapy with low intensity laser on local and systemic immunomodulation following focal brain damage in rat. J Photochem Photobiol; B 97: 145-151; Wu X, et al. (2009) 810 nm Wavelength light: an effective therapy for transected or contused rat spinal cord. Lasers Surg Med 41: 36-41; Fitzgerald M, et al. (2010) Near infrared light reduces oxidative stress and preserves function in CNS tissue vulnerable to secondary degeneration following partial transection of the optic nerve. J Neurotrauma; 27: 2107-2119.)

Delivery of R/NIR-IT following acute contusion TBI has been found to result in significant improvements in Neurological Severity Scores in animals treated with 665 nm and 810 nm R/NIR-IT. (Wu Q, et al. (2012) Low-level laser therapy for closed-head traumatic brain injury in mice: effect of different wavelengths. Lasers Surg Med; 44: 218-226.) Significant effects of exposure to NIR light during recovery have also been seen in additional animal studies, with observations representing a picture of a more active and goal-seeking animal after exposure to NIR. (Quirk BJ, et al. (2012). Near-infrared photobiomodulation in an animal model of traumatic brain injury: improvements at the behavioral and biochemical levels. Photomed. Laser Surg.; 30(9): 523-529.)

In certain embodiments, the invention would be added at the time of decompressive craniectomy and the opening of dura. The NIR grid light sources would be placed directly onto the surface of brain tissue at risk and may be secured by stitching to the dura above it (brain-NIR grid-dura). Grids may be constructed in different shapes and dimensions allowing for customization of their application to cover the target tissue . This may be analogous to placing electrode grids used for mapping out epileptic foci of the brain. The leads may be brought out through the dura and skin and attached to appropriate hardware.

In another embodiment, the NIR light source can be directly applied to the brain surface, introduced through ports in the adhesive surgical brain balloon or bag.

Variables in treatment include irradiation sources (prior to the instant invention, limited to laser or light-emitting diode), mode of delivery (pulsed or continuous), stimulation wavelength (630, 670, 780, 810, 830, 880 or 904 nm), total dose (i.e., joules of irradiation per unit area), rate of delivery of the irradiation energy [watts per unit area (note: watts=joules °ø time), also referred to as fluence], duration (length of exposure), timing (pre- or post-insult), depth of a target cell, continuous wave or pulsed mode, pulse parameters and frequency of treatment (Quirk and Whelan, 2011). (See also, e.g., Karu IT, Low-Power Laser Therapy”, in Biomedical Photonics Handbook, \To-Dinh T. Ed., CRC Press, Boca Raton, FL, pp. 48-1 to 48-25, (2003) incorporated herein by reference)

In certain embodiments, grids would be left in place as long as patient has signs of brain swelling and neurological dysfunction. Days or even weeks. Other applications might require more permanent placements for events such as seizures, severe brain injury, etc.

In addition to surface grid NIR sources, depth light sources might be employed to reach deeper injuries. These, again, are analogous to depth electrodes used for seizure mapping—thin tubes containing NIR LEDS are stereotactically inserted into target tissue to deliver NIR to deep brain matter not adequately illuminated by surface grids.

These NIR constructs could be low profile, very low heat emitting, soft and flexible, and biocompatible. They may be sterilized by Gamma radiation, sterilization baths, etc. They may be reusable. In certain embodiments, they may also carry diagnostic components to measure in real time, ATP production, oxidative states of the mitochondrial cytochromes (COX), and perhaps other markers of the traumatic brain extracellular milieu—lactic acid, pH, excitatory amino acids such as glutamate, etc. These diagnostic probes would guide treatment and define optimal NIR protocols, individualized for each patient.

In another embodiment, therapeutic molecules such as central nervous system-specific growth factors, therapeutic biologics, small molecule drugs, nutrients, neurotransmitters, chemotherapeutic agents, therapeutic viruses, nanoparticles, and cell-based therapeutics can be introduced directly to the brain tissue through ports in the adhesive surgical brain balloon or bag or infused through channels engineered directly into the grids.

In another embodiment, ports in the surgical brain balloon or bag can be used to serially sample the extracellular milieu in order to evaluate the efficacy of therapeutics or other parameters such as lactate, glucose, gluconate, pH, CO2, O2, and intracellular proteins. Channels engineered directly into the grids could also be used to sample the extracellular milieu, providing a more topographic representation.

In other embodiments, the surgical brain balloon or bag according to the invention is portable to enable use by first responders. In these embodiments the introduction through the ports of procoagulants, cooling systems or agents and anti-septic agents and mechanisms will be particularly important. This portable bag system could be employed by first responders as a miniature sterile operating field allowing for the execution of minor emergency procedures.

In another embodiment, temperature regulation of the central nervous system including induction of therapeutic hypothermia can be performed.

The invention is further illustrated by the following non-limiting examples.

### EXAMPLES

### Example 1

A teenage male was the unrestrained passenger of high speed motor vehicle accident and was ejected from car. He was found pulseless and without spontaneous respirations by paramedics. He was intubated, provided with CPR on the scene and arrived at receiving trauma room with:
a) Pulse but no spontaneous respirations;
b) Contusions and abrasions noted on head and body;
c) No eye opening spontaneously or to stimulation;
d) Right pupil 5 mm, left 3 mm, poorly reactive;
e) No spontaneous movements, or withdrawal to pain;
f) Glasgow Coma Scale 3T (3-15).

A head CT reveals 2 cm right frontal-parietal acute subdural hematoma (traumatic brain bleed) with 5 cm midline shift (sufficiently large to shift brain right to left). The remainder of trauma survey was negative except for closed femur fracture. The diagnosis was severe traumatic brain injury, GCS 3T. Under the current state of the art management he would receive emergency decompressive craniotomy to drain acute subdural hematoma. A ventriculostomy catheter would be inserted (tube inserted into ventricle fluid space within to measure subsequent intracranial pressure) and a Licox brain probe would be placed to measure temperature, oxygen tension of tissue adjacent to probe. At 38 hours postoperatively, the patient had out of control ICP, remaining>25 mmHG and flap bulging. Patient is placed into phenobarbital coma and body cooled to 32° C. (both interventions have minimal clinical data support, but represent last-ditch efforts at many institutions). At 48 hours postop the ICP>30 mmHg and CPP<40, Licox<10, pupils 6 mm, unreactive. A repeat CT shows massive brain swelling and herniation syndrome. At 96 hours postop, patient declared legally brain dead and the family agrees to withdraw life support and consent to organ donation.

Alternative possible management method according to the present invention:

Steps:
a) Emergency expanded decompressive craniectomy (save and freeze the bone);
b) Evacuate SDH;
c) Place Ventriculostomy;
d) Place Licox;
e) Apply the surgical neurological balloon of the instant invention and attach to artificial CSF recirculation unit; and
f) Patient maintained on pharmacological paralysis and sedation.

Next, immediate postop it is expected that the patient's brain is visible, soft with good pulsations, ICP 2-5 mmHg; CPP 70; Licox 20; brain temp 37C.; CSF should be sent for analysis of Excitotoxins (baseline) and metabolites: glucose, lactate, glutamate, pO2, pCO2, pH. At 24 hours postop it is expected that the pupils will be equal, but minimally reactive, brain swollen, expanding above borders of craniectomy cuts, pulsatile, ICP 5-10 mmHg, CPP 70 mmHG, Licox 25, brain temp via CSF recirculator to 32° C., body temperature at 37° C., and CSF sampling should be done at 6-hour intervals for evaluation of excitotoxins. At 38 hours postop the pupils should be 4 mm and slowly reactive. The brain further swollen, extending ˜1 cm beyond normal vault volume, ICP 5-10 mmHg, CPP 70 mmHg, Licox 25, brain temperature maintained via recirculator at 32° C., and body temp 37° C. with CSF sampling continues to monitor brain metabolites and excitotoxins.

At 7 days postop, the pupils should be 4 mm with brisk reactivity, the brain swelling beginning to resolve, Intracranial pressure (ICP) remains 5-10 mmHg, cerebral perfusion pressure (CPP) remains>70, Licox progressively rising, brain temperature slowly normalized, as tolerated by ICP/CPP, CSF sampling continues. At 10 days postop, the patient is expected to be returned to the OR for removal of surgical neurological balloon and closure of scalp. The ventriculostomy and Licox remain, but are removed as measurements normalize. The patient's pharmacological paralysis and sedation are slowly discontinued.

At 4 weeks postop, the patient is expected to return to the OR for autologous cranioplasty (replacement of his skull) and is referred to rehabilitation center for further recovery with no further neurosurgical intervention.

The disclosure of all publications, including patents, and patent applications, cited in this specification are hereby incorporated herein by reference in their entirety for the proposition for which they are disclosed.

Having described the invention in detail and by reference to the preferred embodiments thereof, it will be apparent that modifications and variation are possible without departing from the scope of the following claims.

1. A surgical device comprising a temporary flexible covering for an exposed brain or spinal cord that allows the brain or spinal cord to swell following injury.

2. The device according to claim 1 that allows for almost total exposure of the brain and affected area(s) of the spinal cord into a therapeutic, decompressive, monitored, sterile environment that is amenable to additional therapeutic intervention.

3. The device according to claim 1 further comprising at least one access port.

4. The device according to claim 3 further comprising a single use, disposable device with multiple accessible ports for monitor tubing and electrical cables, for infusion, for sampling of extracellular milieu, for a fluid recirculator, or for one or more other medical devices.

5. The device according to claim 4 wherein the other medical device emits energy.

6. The device according to claim 5 wherein the energy is light emitted is in the infrared or near infrared wave lengths.

7. The device according to claim 4 wherein the other medical device is magnetic, emits radiation, or comprised of ultrasonic waves.

8. The device according to claim 4 wherein the other medical device provides directed therapeutic temperature regulation including induced hypothermia.

9. The device according to claim 4 wherein the at least one port infuses medication selected from antibiotics, saline solution, central nervous system-specific growth factors, therapeutic biologics, small molecule drugs, nutrients, neurotransmitters, chemotherapeutic agents, therapeutic viruses, nanoparticles, and cell-based therapeutics.

10. The device according to claim 1 wherein the device is constructed from soft, relatively translucent biocompatible material.

11. The device according to claim 10 wherein the device allows for long term maintenance of completely sealed and sterile environment which removes volume expansion restrictions for brain and spinal cord swelling.

12. The device according to claim 10 wherein the device allows for previously unprecedented serial sampling of extracellular milieu to evaluate baseline and evolving metabolic consequences of brain and spinal cord injury as well as feedback on interventions.

13. The device according to claim 1 wherein the temporary covering is maintained on the patient for less than one year.

14. A method for using the device according to claims 1 through 13 comprising performing a surgical procedure.

15. A method of removing accumulated proteins and or toxins using the device according to claims 1 through 13 comprising washing the central nervous system.

16. The method according to claim 14 wherein the brain is washed with antiseptic fluid that is free from harsh detergents and unwanted proteases.

17. The method according to claim 14 wherein the fluid further comprises beta amyloid specific antibodies.

18. The method according to claim 14 wherein the fluid further comprises coated magnetic particles.

19. The method according to claim 14 wherein the washing is done to prevent or treat neurodegenerative diseases.

20. The method according to claim 18 wherein the disease is Alzheimer's disease.

21. The method according to claim 18 wherein the disease is Huntington's disease.

22. The method according to claim 18 wherein the disease is Parkinson's disease.

23. The method according to claim 18 wherein the disease are prion diseases or transmissible spongiform encephalopathies such as Creutzfeldt-Jakob Disease.

24. The method according to claim 18 wherein the disease is chronic traumatic encephalopathy or chronic trauamatic encephalomylitis.

25. The method according to claim 18 wherein the disease is congenital metabolic such as mucopolysaccaridosis.

26. The method according to claim 18 wherein the disease is demyelinating or autoimmune such as multiple sclerosis or lupus cerebritis.

27. The method according to claim 14 wherein the method involves cerebral therapeutic hypothermia.

28. The method according to claim 14 wherein the fluid further comprises beta amyloid specific antibodies.

29. The method according to claim 18 wherein the disease is includes brain or spinal cord tumors.
